(19) United States
(12) Patent Application Publication
(10) Pub. No.: US 2011/0040239 A1
(43) Pub. Date: Feb. 17, 2011

(54) APPARATUS FOR THE PRODUCTION OF IONIZED OXYGEN AND OZONE FROM PURE OXYGEN AND METHODS FOR USING SAME IN MEDICAL APPLICATIONS

(76) Inventors: Hans-Georg Schneider, Berlin (DE); Oliver Schneider, Berlin (DE); Helmut Zimmermann, Sispony (AD); Klaus Dieter Zimmermann, Erkelenz (DE)

Correspondence Address: R. MICHAEL WEST;LAW OFFICES OF R. MICHAEL WEST, 1922 21ST STREET, SACRAMENTO, CA, 95811, US

(21) Appl. No.: 12/539,469

(22) Filed: Aug. 11, 2009

(51) Int. Cl. A61M 37/00; B01J 19/08; G05D 7/00

(52) U.S. Cl. 604/25; 422/186.08; 422/111

(57) ABSTRACT

An apparatus for the production of ionized oxygen and ozone from pure oxygen. An adjustable high voltage power supply is connected to an ozone generator having at least one ozone generator therein. The HV power supply has a relatively low voltage setting for producing negative ionized oxygen and a relatively high voltage setting for producing ozone. A negative ionizer may be included to increase the concentration of negative ionized oxygen. Outputted gasses are directed to a hermetically sealed envelope positioned around and in spaced relation from the surface of a patient's injury. If the wound is infected, ozone is selectively used to treat the infection for a first predetermined period of time sufficient to neutralize the infection. After the wound is treated with ozone, negative ionized oxygen is selectively used to treat the wound for a second predetermined period of time sufficient to enhance the healing of the wound.

### BACKGROUND OF THE INVENTION

1. Field of the Invention

The invention relates generally to devices for selectively modifying pure oxygen, either into ozone or into an ionized form, and to methods for selectively using those gases in treating infected wounds and in enhancing the speed and effectiveness of wound healing. More specifically, the invention pertains to a compact and portable system for generating both ozone and ionized oxygen on demand, and alternatively or successively delivering these gases to a hermetically sealed envelope surrounding a patient's wound.

2. Description of the Prior Art

Ozone (O₃) has been used for many years as a disinfectant for laundry, water, air, food, room surfaces, and other objects contaminated by bacterial or viral agents. In medical applications, ozone has been used to disinfect operating rooms and surgical instruments, and for direct application on patients' wounds. For example, in U.S. Pat. No. 6,073,627, issued to Sunnen, an ozone generator provides controlled amounts of ozone to an exposed infected or poorly healing portion of a patient's body. Sunnen's system employs a coronal discharge generator and various ozone, humidity, and temperature sensors to oversee application of a combination of pure oxygen and ozone gas to the patient's wound.

O₃ is an allotrope of oxygen that is much less stable than O₂, the diatomic form of oxygen. Ozone cannot effectively be stored, because in less than an hour from the time it is generated, most of the ozone has reverted to O₂. Therefore, as a practical matter, ozone must be generated shortly before or as it is being utilized. To that end, a number of methods for generating ozone on location have been developed, using either ambient air or pure oxygen to produce the desired quantity and concentration of ozone.

A first method relies upon the spark of corona discharge, produced by a high voltage applied to a corona discharge tube. U.S. Pat. No. 6,019,949, granted to Dunder, illustrates a Ozone Generator Control System based upon the corona discharge principle. The corona discharge method can be practiced by relatively inexpensive apparatus, and has the additional advantage that ozone may be generated from ambient air. However, the corona discharge method using air as a subject gas produces nitrogen oxides as a by-product, which must be removed from the ozone stream.

A second method of producing ozone employs the irradiation of air or oxygen with ultraviolet light. UV ozone generators are also relatively inexpensive, but produce low concentrations of ozone and require a substantial residence time for the subject gas to produce usable ozone concentrations. Thus, UV ozone generators are not practical where the subject gas is moving quickly, or where the application requires higher ozone concentrations for utilization.

A third method of manufacturing ozone is known as the cold plasma, or dielectric barrier discharge (“DBD”) method. This method acts upon pure oxygen, which is passed over electrodes separated by a dielectric insulator and charged with a high voltage. One apparatus producing ozone using this method is disclosed in U.S. Pat. No. 6,284,205, issued to Murata et al., for an Ozonizing Unit Ozone Generator And Ozone-Processing System.

Another apparatus relying upon the dielectric barrier discharge method is the PATT (“Physical Air Treatment Technology”) Module, developed by ETR PS GmbH, of Dortmund, Germany. The PATT module employs a miniaturized planar module including a grid-work of electrodes energized by carefully controlled high voltage. Fed with a source of pure oxygen, the PATT module technology is capable of producing ozone up to approximately 6% in concentration, from a very compact device.

Other forms of oxygen have also been advocated as providing health benefits. For example, ionized oxygen inhalation therapy (“IO₂IT”) contemplates that a mixture of medical oxygen and selectively negative or positive oxygen ions may be inhaled for therapeutic or preventative purposes. U.S. Pat. No. 5,381,789, issued to Marquardt, discloses an Ionizer For The Ionization Of Oxygen In Oxygen Therapy, to be used for IO₂TT. This device includes ionizing needles charged with a high voltage of approximately 5,500 volts, below the ozone-creating voltage level described as 6,700 volts. Because of its potentially dangerous effects when inhaled by a patient, the production of ozone by the Marquardt device is specifically avoided.

### SUMMARY OF THE INVENTION

The medical apparatus disclosed herein comprises a compact and portable gas generator, having selective adjustments to output either ozone or ionized oxygen in medically efficacious quantities. The ozone and ionized oxygen are used to treat a patient's skin wounds, such as lesions and bums, by eliminating infections and accelerating the rate of healing.

Pure oxygen is fed at a predetermined rate into an ionization chamber within the apparatus, where one or more dielectric barrier discharge modules resides. High voltage is delivered to the electrodes of the modules at a voltage calculated to produce either a relatively high concentration of ozone in a first mode of operation, or a relatively high concentration of ionized oxygen in a second mode of operation.

The gas outputted from the ionization chamber is passed through a negative ionizer, to increase the concentration of negatively ionized oxygen, in the second mode of operation. The gas outputted from the negative ionizer through a gas treatment line may be humidified before utilization. A humidifier particularly enhances the longevity and effectiveness particularly of ozone gas. A heater is also provided in the gas treatment line, to raise the temperature of the outputted gas as necessary for the comfort of the patient.

A three-way valve is located within the gas treatment line, downstream from the generator and the heater. In a closed position, a three-way valve isolates the gas treatment line from any further distribution of the outputted gas. This position may be selected as a start up position, or a shut down position, for the apparatus.

The three-way valve also has a first open position, where the valve directs the outputted gas to a hermetically sealed envelope, arranged around and in spaced relation from an injury or wound on the patient's skin. The envelope may assume a wide variety of sizes and configurations, depending upon the location and nature of the patient's injury or wound. But in all cases, the envelope acts to confine the gas outputted from the ionization chamber and the negative ionizer and direct it to the vicinity of the injury or wound. Because the envelope is spaced from the injury or wound, it will not aggravate it through mechanical abrasion or other physical contact.

The sealed envelope also has a gas discharge line, connected to an input of an ozone converter. The ozone converter, typically using a catalytic element, transforms the O₃ into O₂, which can safely be discharged into the ambient air of the treatment room.

A selectively operable vacuum pump may also be provided in the discharge line from the ozone converter. This vacuum pump may be actuated at the end of a treatment session, to facilitate removal of ozone and wound foam from the sealed envelope.

The three-way valve additionally has a second open position, where either ozone or ionized oxygen is by-passed into another input of the ozone converter, for direct conversion into O₂ This position maybe used, for example, when calibrating the apparatus, or when switching from the production of ozone to ionized oxygen, or visa-versa.

Predetermined treatment regimes for successive use of ozone and then ionized oxygen, for eliminating infection and then enhancing the healing process, may be employed through appropriate control of the amount and type of gas outputted from the apparatus.

### BRIEF DESCRIPTION OF THE DRAWINGS

FIG. 1 is a diagrammatic representation of an ozone and ionized oxygen generator and associated components used for selectively delivering those gases to a patient's wound;

FIG. 2 is a more detailed depiction of the ionization chamber and the plurality of dielectric barrier discharge modules therein;

FIG. 3 is a graph depicting the relationship between the variable high voltage delivered to the dielectric barrier discharge modules, and the quantity of either ozone or ionized oxygen outputted therefrom;

FIG. 4 is a representation of the molecular creation of ozone from pure oxygen;

FIG. 5 is a depiction of an alternative construction of a hermetically sealed circular envelope used to apply gases to a large surface wound on the upper torso of a human;

FIG. 6 is a depiction of a hermetically sealed oval envelope used to apply gases to a neck wound; and,

FIG. 7 is a fragmentary depiction of an alternative construction of the hermetically sealed envelope used to apply gases to extensive foot or leg wounds.

### DETAILED DESCRIPTION OF THE PREFERRED EMBODIMENT

Turning now to the drawings, the gas generation apparatus 11 comprises a controller 12 having an oxygen flow rate meter 13 and a voltmeter 14. Tank 16 provides a convenient and ready source of pure oxygen, O₂, for utilization by the apparatus 11 particularly in a home setting. Pure oxygen may also be supplied from a fixed oxygen valve provided in the wall of a treatment room in a hospital or a clinic. An oxygen supply line 17, extends between tank 16 and an inlet 18 at one end of ionization chamber 19.

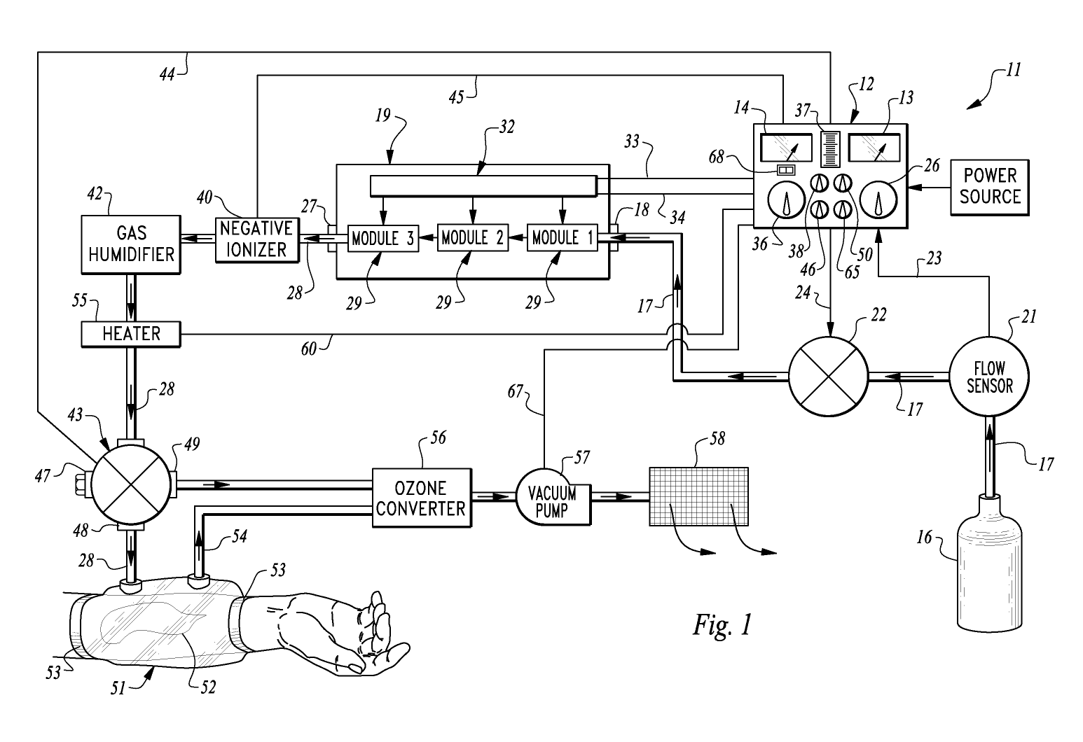

An oxygen flow rate sensor 21 and an electronically controlled valve 22, are interposed in supply line 17, as shown in FIG. 1. Sensor line 23 delivers the electronic data regarding oxygen flow through line 17 to controller 12. Valve control line 24 delivers opening and closing electronic signals to valve 22. Oxygen flow rate meter 13 provides an instantaneous indication of the rate at which oxygen is flowing through line 17. By rotating flow control knob 26 in a selected direction, valve 22 is either opened or closed to the desired extent, so that the desired oxygen flow rate is achieved. As will be explained more fully below, the rate at which oxygen is passed through the apparatus 11 has a direct impact on the concentration of ozone or ionized oxygen produced, and ultimately administered to a patient's wound.

Line 17 is interconnected to an inlet 18, provided at one end of ionization chamber 19. An outlet 27 is provided at the chamber's other end, remote from inlet 18. Outlet 27 in turn, is connected to a treatment gas line 28. As shown in FIG. 1, ionization chamber 19 includes a plurality of ozone generators 29 therein, serially arranged between inlet 18 and outlet 27. Although at least one ozone generator 29 is required to practice the invention, it is preferred that a plurality of ozone generators 29 be employed to increase the concentration of ozone and ionized oxygen produced by the apparatus 11.

It is also preferred that the ozone generator be of the Dielectric Barrier Discharge (“DBD”) design, which lends itself to a compact configuration and provides efficient ozone production. Applicants further prefer for their ozone generators 29, the PATT (“Physical Air Treatment Technology”) miniaturized planar modules, manufactured by ETR PS GmbH, Altwickeder Hellweg 195, D-44319 Dortmund, Germany. These modules are extremely small in size, compared to the prior art Siemens Tube previously used to produce ozone. Through the use of these miniaturized modules, the ionization chamber 19 and the apparatus 11, can be made compact and portable, allowing physical treatments to be provided for patients wherever it is convenient, including their homes.

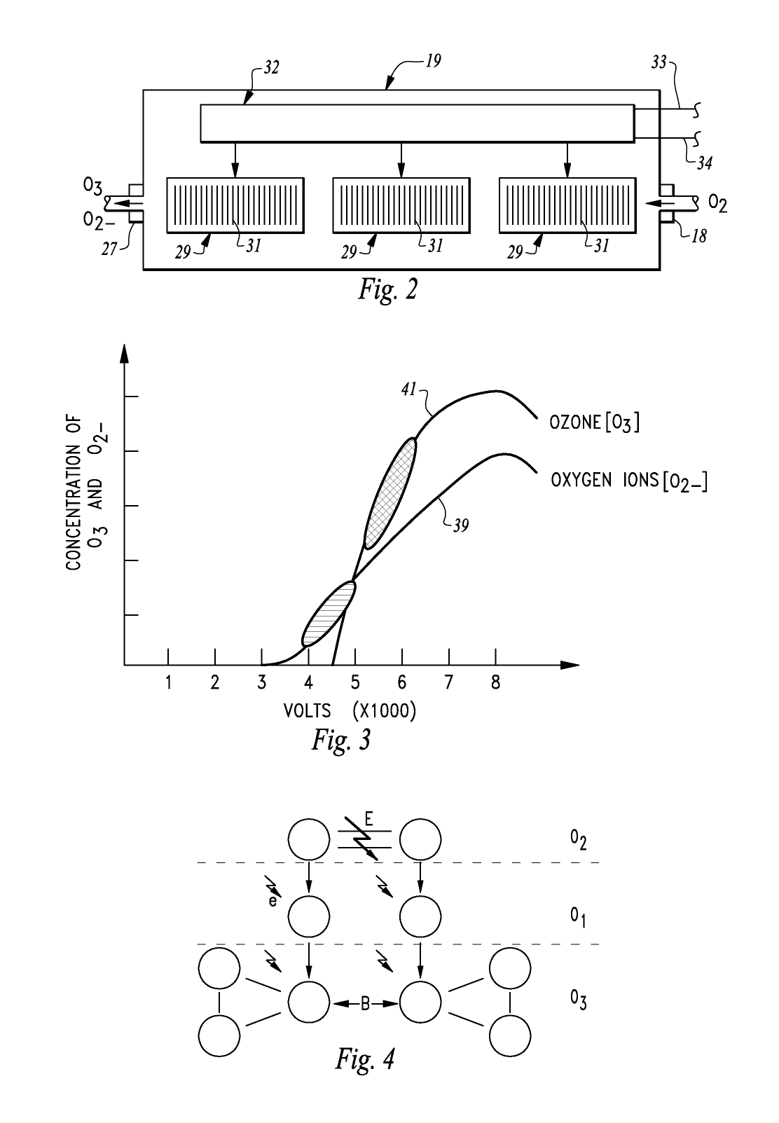

As shown in FIG. 2, ozone generators 29 include a plurality of electrodes 31 which are provided with High Voltage alternating current from regulator assembly 32. An HV line 33 and a ozone sensor line 34 interconnect regulator assembly 32 with the controller 12. HV knob 36 provides continuous adjustment of the High Voltage applied to the electrodes 31, and voltmeter 14 provides a continuous readout of that High Voltage. Controller 12 also includes an LED bar graph 37, and an ozone adjustment knob 38. Bar graph 37 provides an instantaneous readout of the ozone concentration present in ionization chamber 19, and adjustment knob 38 in combination with microprocessor controller provides the means to raise or lower that concentration. It may also be desirable to have additional ozone concentration sensors downstream from ionization chamber 19, to monitor the overall parameters particularly of ozone production. Typically, in a first mode of operation, when ozone is being produced in the ionization chamber 19 owing to the appropriate adjustment of HV adjustment knob 36 and adjustment knob 38, the concentration of ozone will be in the range of 1% to 6%, or so.

The basic process used to manufacture ozone on demand is illustrated in FIG. 4. As a first step, laboratory grade oxygen O₂ is transformed into O₁, owing to the molecular splitting forces imposed by the electric force E. The O₁ joins with remaining O₂ molecules through the bonding process B, to form ozone O₃.

FIG. 3 depicts how varying the HV voltage applied to the electrodes of the ozone generators 29 changes the gases outputted by ionization chamber 19. At lower voltages, from approximately 3.9 KV to 5.0 KV, negatively ionized oxygen, O₂, is produced in relatively high concentrations. This function is represented by graphed line 39. At the relatively high voltage setting from approximately 5.1 KV to 6.2 KV, ozone is produced in relatively high concentrations. This function is represented by graphed line 41. Thus, by varying HV adjustment knob 36 and being mindful of voltmeter 14, the ionization chamber 19 can selectively be programmed to produce higher concentrations of either negatively ionized oxygen or ozone. Other adjustment means, such as ozone adjustment knob 38 and flow control knob 26, are also effective in predetermining and maintaining the desired concentration and type of gases outputted from ionization chamber 19.

Immediately downstream from ionization chamber 19, treatment gas line 28 delivers the outputted gases to negative ionizer 40, of conventional design. Ionizer 40 includes an active electrode, provided with high voltage through ionizer control line 45. The electrode included in the ionizer 40 may be of varied configurations, including a needle, a bar, a ring, a plate, or a ball. The principal function of ionizer 40 is to increase the concentration of negatively ionized oxygen, compared to positively ionized oxygen outputted from chamber 19, when the apparatus is programmed to produce ionized oxygen in a second mode of operation of apparatus 11.

Downstream from ionizer 40 is gas humidifier 42. It is well recognized that increasing the humidity of ozone, will increase both its longevity and its efficacy in killing bacterial and viral populations. Treatment gas line 28 resumes downstream from gas humidifier 42, delivering outputted gases to a heater 55. The temperature of outputted gases maybe uncomfortably cool, so heater 55 through power supplied by heater control line 60, raises the gas temperature as needed for the comfort of the patient.

The output from heater 55 is delivered through treatment gas line 28 to valve means 43. Preferably, valve means 43 is a three-way valve, so that outputted gases can conveniently be directed to different ports, for start up, operation, and shut down. Valve control line 44 provides an electrical interconnection between valve means 43 and controller 12, so that the position of valve means 43 can be determined by the rotational position of valve control knob 46 on controller 12.

Valve means 43 includes a plugged port 47, a first discharge port 48 and a second discharge port 49. In a closed position directed toward port 47, valve means 43 isolates the gas treatment line 28 from any further distribution of the outputted gas. This position may be selected as a start up position, or a shut down position, for the apparatus 11.

Valve means 43 also has a first open position, in which outputted gases are directed through first discharge port 48 and gas treatment line 28 to a hermetically sealed envelope 51. As shown in FIG. 1, envelope 51 is arranged around a portion of a patient's forearm, and is maintained in spaced relation from an injury or wound 52 on the patient's skin. As will be described herein, the sealed envelope may assume a variety of configurations, depending upon the size, location, and nature of the patient's injury or wound. However in all cases, the sealed envelope acts to confine the gas outputted from the ionization chamber 19, and to direct it toward the patient's exposed injury or wound.

Sealed envelope 51 is preferably substantially transparent and flexible, to facilitate its proper placement around the wound. Because ozone breaks down many materials, causing oxidation and eventual structural failure in some cases, envelope 51 should be constructed of a material which is both transparent and immune from the ill effects of ozone. Envelope 51 also includes elastic and/or hook and loop material around envelope cuffs 53 at an entry end and at an exit end, to provide a snug but comfortable seal so that gases cannot escape from the envelope. The envelope is sized to be spaced from the patient's injury or wound, so it will not aggravate it through abrasion or direct contact.

Sealed envelope 51 also has a gas discharge line 54, connected to an input of an ozone converter 56. Discharge line 54 relieves pressure on sealed envelope 51, and allows the admittance of fresh treatment gases into envelope 51. Ozone converter 56 transforms the waste O₃ outputted from the envelope 51 into O₂. This may be accomplished by means of a catalytic converter, such as that shown identified as element 40 in U.S. Patent Application Publication No. US 2008/0310992 A1. A vacuum pump 57 may be provided at the discharge of converter 56, to draw gases and any wound foam therethrough, for subsequent discharge through grating 58. Pump 57 is powered by pump control line 67, selectively actuated at the end of a treatment session by turning on switch 68. The outputted O₂ can safely be discharged into the ambient air of the treatment room.

Valve means 43 additionally has a second open position, where either ozone or ionized oxygen is by-passed through the second discharge port 49 into another input of the ozone converter 56, for conversion into O₂. This position may be used, for example, when calibrating the apparatus 11, or when switching from the production of ozone to ionized oxygen, or visa-versa.

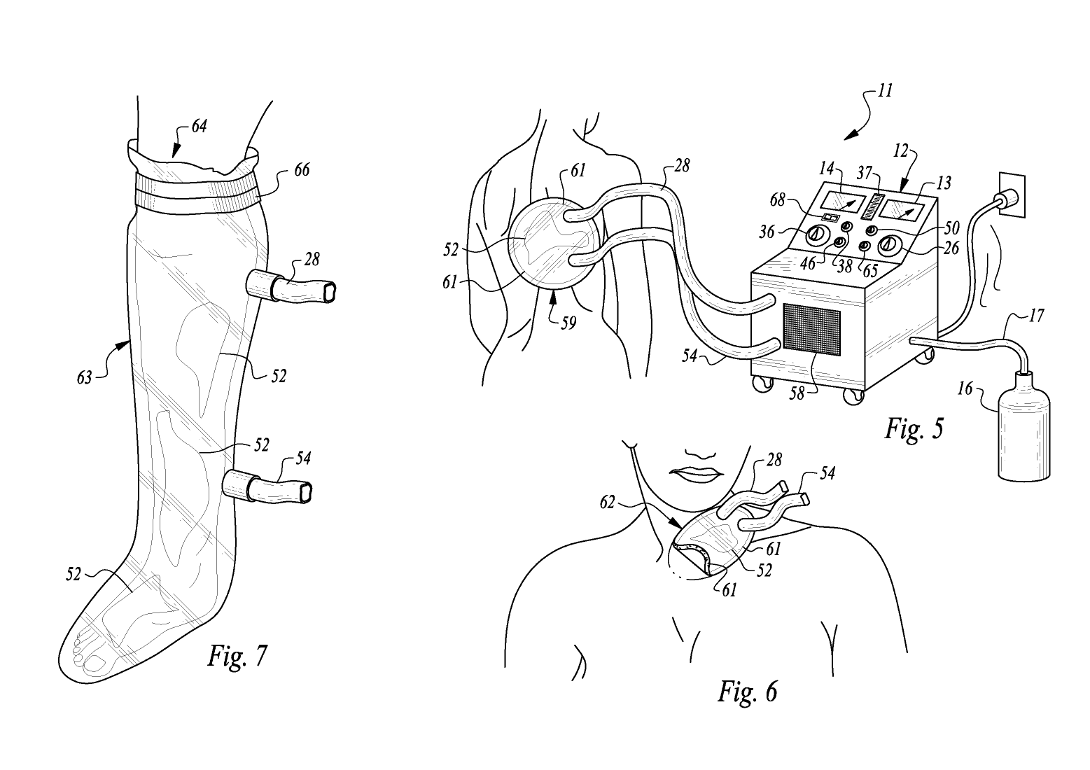

Because wounds and injuries may be of different sizes and numbers, and located in different locations on the patient's body, the hermetically sealed envelope used for a particular treatment may assume different configurations and structural features. For example, in FIG. 5, sealed envelope 59 is comprised of a circular sheet of material, adapted to fit over an injury 52 on the patient's back. Treatment gas line 28 and gas discharge line 54 are provided as previously described. Sealed envelope 59 has a lower side and a strip of adhesive 61 extending around the periphery of this lower side for temporary affixation to the patient's back during treatment. The adhesive is sufficient to provide a hermetical seal around the envelope 59, to confine treatment gases therein.

Another adaptation of this design is sealed envelope 62, shown in FIG. 6. In this instance, the patient's injury 52 is on her neck. By manufacturing envelope 62 in an oval configuration, and providing the same adhesive strip 61 around the periphery of the lower side of the envelope, the envelope 62 may be applied directly around the affected portion of the patient's neck. Treatment gas line 28 and gas discharge line 54 are included to provide gas input and output functions as with the other embodiments.

Lastly, FIG. 7 shows a sealed envelope 63 sized and configured to treat multiple injuries 52 on the lower leg and foot. Sealed envelope 63 has a single opening 64 at its upper end, including a circumferential elastic band 66 to provide a hermetical seal with the patient's skin. The lower end of envelope 63 is sealed and configured to surround the patient's foot like a loose sock. Treatment gas line 28 located adjacent the upper end of envelope 63 provides for ingress of treatment gases, and gas discharge line 54 located adjacent the ankle portion of envelope 63 provides for egress of treatment gases.

In practicing the method for treating wounds of the present invention, a source of pure oxygen is provided. Next, the pure oxygen is continuously ionized with either a relatively high voltage in a first mode of operation to produce ozone, or continuously ionized at a relatively low voltage in a second mode of operation to produce negatively ionized oxygen. In both cases, the gas produced by the apparatus 11 is delivered to a patient's wound, and confined within a volume above and to an area around the wound.

Typically, ozone would first be applied for a predetermined amount of time to the patient's wound, sufficient to neutralize the infection, killing bacterial or viral agents. This period of time may be from 10 to 30 minutes, or so. Then, the negatively ionized oxygen would be applied for a predetermined amount of time to the wound, sufficient to enhance the healing of the wound. This second predetermined amount of time may also be from 10 to 30 minutes, or so. But the precise amount of time for each treatment will be ascertained by medical personnel, taking into consideration the nature and extent of the injury and any associated infection.

It is apparent that multiple treatments over the course of a day, weeks, or months may be necessary, to effect full healing of a particular wound. It is also apparent that the first predetermined period of time for treatment with ozone, need not necessarily be the same as the second predetermined period of time for treatment with negatively ionized oxygen, and that in a given situation, only one of the treatments may be necessary. But one of the features of the apparatus 11, and the methods of using same, is that both of the gases may be applied successively and alternatively generated and applied to the patient from a single apparatus, as needed, for the most efficacious results for a patient.

What is claimed is:

1. An apparatus for the production of ionized oxygen and ozone from pure oxygen, comprising:
a. a source of pure oxygen, said source including an oxygen supply line;
b. an ionization chamber, said ionization chamber including an inlet at one end, said inlet being connected to said oxygen supply line, and an outlet at the other end, said outlet being connected to a treatment gas line, said chamber including at least one ozone generator therein, between said inlet and said outlet;
c. an adjustable high voltage power supply, said power supply having an output connected to said ozone generator, said power supply having a relatively low voltage setting for producing negative ionized oxygen and a relatively high voltage setting for producing ozone;
d. ozone converting means for transforming ozone into oxygen, said converting means having a first input, a second input, and an output in communication with the ambient air;
e. a hermetically sealed envelope in communication with said treatment gas line and having a discharge line in communication with said second input; and,
f. valve means for selectively directing gas passing through said treatment gas line alternatively to said sealed envelope or to said first input of said ozone converting means.

2. An apparatus as in claim 1 in which said ozone generator is a dielectric barrier discharge module.

3. An apparatus as in claim 2 in which the relatively low voltage setting for producing ionized oxygen is generally within the range of 3.9 KV to 5.0 KV, and in which the relatively high voltage setting for producing ozone is generally within the range of 5.1 KV to 6.2 KV.

4. An apparatus as in claim 2 including a control valve and a flow rate sensor in said oxygen supply line, and further including control means to monitor an electrical output of said flow rate sensor and to adjust the position of said valve to establish a predetermined rate of oxygen flow through said supply line.

5. An apparatus as in claim 2 further including a gas humidifier in said gas treatment line, between said outlet and said valve means.

6. An apparatus as in claim 5 further including a negative ionizer in said gas treatment line, between said ionization chamber and said gas humidifier.

7. An apparatus as in claim 5 further including a heater in said gas treatment line, between said gas humidifier and said valve means.

8. An apparatus as in claim 2 in which said valve means comprises a three-way valve, having an off position in which flow of gas through said gas treatment line is prevented.

9. An apparatus as in claim 2 including a plurality of said dielectric barrier discharge modules, each of said modules acting together to increase the volumetric percentage of either negative ionized oxygen or ozone, depending upon the voltage setting of said high voltage power supply.

10. An apparatus as in claim 2 in which said sealed envelope is flexible and substantially transparent.

11. An apparatus as in claim 10 in which said sealed envelope has an entry end and an exit end, for the insertion of a human appendage therethrough.

12. An apparatus as in claim 11 further including means for hermetically sealing said entry end and said exit end around a human appendage.

13. An apparatus as in claim 10 in which said sealed envelope comprises a sheet of material, said sheet having a lower side and adhesive means extending around a periphery of said lower side for hermetically sealing with human skin.

14. A method for treating wounds, comprising the steps of:
a. providing a source of pure oxygen;
b. continuously ionizing said oxygen with either a relatively low voltage to produce negative ionized oxygen or a relatively high voltage to produce ozone; and,
c. delivering either said negative ionized oxygen or said ozone to a wound, and confining said negative ionized oxygen or said ozone to a volume above and to an area around the wound.

15. A method as in claim 14 in which the wound is infected, and in which ozone is delivered to and confined around the wound to treat the infection for a first predetermined period of time sufficient to neutralize the infection.

16. A method as in claim 15 in which said first predetermined period of time is approximately ten (10) to thirty (30) minutes.

17. A method as in claim 15 in which negative ionized oxygen is delivered to and confined around the wound after the wound is treated with ozone for a second predetermined period of time sufficient to enhance the healing of the wound.

18. A method as in claim 17 in which said second predetermined period of time is approximately ten (10) to thirty (30) minutes.

19. A method for treating wounds, comprising the steps of:
a. providing a source of pure oxygen;
b. continuously ionizing said oxygen with a relatively high voltage to produce ozone;
c. delivering said ozone to a wound, and confining said ozone to a volume above and to an area around the wound for a first predetermined period of time;
d. continuously ionizing said oxygen with a relatively low voltage to produce negative ionized oxygen; and,
e. delivering said negative ionized oxygen to a wound, and confining said negative ionized oxygen or said ozone to a volume above and to an area around the wound for a second predetermined period of time.

20. A method as in claim 19 in which said first predetermined period of time is sufficient to neutralize the infection.

21. A method as in claim 19 in which said second predetermined period of time is sufficient to enhance the healing of the wound.
